(19) United States
(12) Patent Application Publication
(10) Pub. No.: US 2026/0191840 A1
(43) Pub. Date: Jul. 9, 2026

(54) TREATMENT OF MIGRAINE

(71) Applicant: Allergan Pharmaceuticals International Limited, Dublin 17 (IE)

(72) Inventors: Joel Trugman, Hoboken, NJ (US); Michelle Finnegan, Madison, NJ (US)

(21) Appl. No.: 19/266,689

(22) Filed: Jul. 11, 2025

Related U.S. Application Data
(63) Continuation of application No. 18/969,499, filed on Dec. 5, 2024, now abandoned.
Application No. 18/969,499 is a continuation of application No. 17/445,463, filed on Aug. 19, 2021, now abandoned.
Application No. 17/445,463 is a division of application No. 16/433,200, filed on Jun. 6, 2019, now abandoned.
(60) Provisional application No. 62/682,345, filed on Jun. 8, 2018.

(51) Int. Cl. A61K 31/437; A61K 31/404; A61P 25/06

(52) U.S. Cl. CPC A61K 31/437; A61K 31/404; A61P 25/06

(57) ABSTRACT

The application provides methods for the treatment of migraine for patients that are poor responders to triptan treatments. Some embodiments provide methods for treating or reducing migraine in patients that do not adequately respond to triptan treatments comprising the step of administering an effective amount of CGRP antagonists; for example, ubrogepant or atogepant, or a pharmaceutically acceptable salt, ester or prodrug thereof.

### FIELD

The application is related to medicaments and methods for treating migraine in patients that are poor responders to triptan treatments.

### BACKGROUND

Migraine is a highly prevalent, severe, and disabling neurological condition with a significant unmet need for effective treatments. (Holland, P. R. & Goadsby, P. J. Neurotherapeutics (2018). The serotonin (5-hydroxytryptamine [5-HT]) receptor subtype 1_{B}/1_{D} agonists, called triptans, are the first-line acute therapy for patients who experience moderate-to-severe migraine attacks. However, a high percentage of patients are not satisfied with this acute treatment, either for lack of response or side effects. (Negro A., et. al., Journal of Pain Research 11 515-526, 2018). In addition, the commonly used triptan class of compounds are ineffective in many patients. Viana M., et. al., reports that about 30% to 40% of patients not responding adequately to triptan therapy. (Cephalalgia 33 (11) 891-896, 2013). Different approaches can be taken to try to improve the intra-individual consistency of response to oral triptans in migraineurs. For the subgroup of patients who despite these attempts do not respond to a particular triptan, the poor responsiveness is likely to be consistent in subsequent attacks. (Dahlof C G H, Cephalalgia, 26 (2) 98-106, 2005). While triptans can be a valuable option for acute treatment of migraine, studies have shown that treatment persistence is low and that there is a significant unmet clinical need despite the wide availability of triptans. (Messali A J et. al., Headache 54 (7) 1120-30, 2014) There is an urgent need to provide effective treatments for patients that do not respond adequately to triptan therapy.

### SUMMARY

The application provides methods for the treatment of migraine for patients that are poor responders to triptan treatments. Some embodiments provide methods for treating or reducing migraine in patients that do not adequately respond to triptan treatments comprising the step of administering an effective amount of CGRP antagonists; for example, ubrogepant or atogepant, or a pharmaceutically acceptable salt, ester or prodrug thereof.

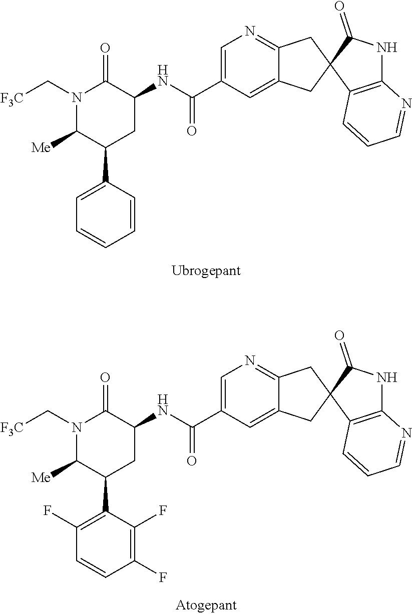 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F ; C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

### DETAILED DESCRIPTION

The application provides methods for the treatment of migraine for patients that are poor responders to triptan treatments. In some embodiments, the poor responders are patients that are non-responders, infrequent responders or insufficient responders to one or more triptan migraine treatments. Some embodiments provide methods for treating or reducing migraine in patients that do not adequately respond to triptan treatments comprising the step of administering an effective amount of a CGRP-antagonist. In a preferred embodiment, the CGRP-antagonist is ubrogepant or atogepant, or a pharmaceutically acceptable salt, ester or prodrug thereof.

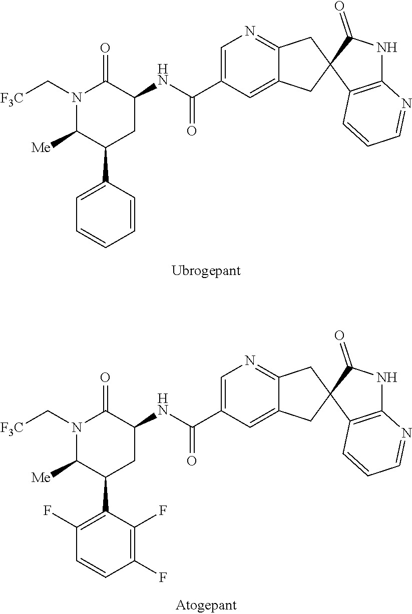 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F ; C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

Some embodiments provide methods for treating or reducing migraine in patients that do not adequately respond to treatment with one or more triptan drugs, for example rizatriptan, sumatriptan, naratriptan, eletriptan, donitriptan, almotriptan, frovatriptan, avitriptan or zolmitriptan. For example, patients may have poor response to prior treatment with one or more triptans after a period of one, two, three, four, five, six, seven, eight, nine, ten, eleven, twelve weeks or more. The poor response can be characterized as non-response to triptans where the frequency and intensity of migraines are unchanged; or insufficient response where some change in the frequency or intensity of migraine is observed, but is inadequate from a clinical perspective. In some embodiments, the patient suffers from one or more symptoms of migraine selected from sinusitis, nausea, nasopharangytis, photophobia, appetite changes, cognition and concentration difficulties, cold extremities, diarrhea or other bowel changes, excitement or irritability, fatigue, frequent urination, memory changes, weakness, yawning, stretching, seeing bright spots or flashes of light, vision loss, seeing dark spots, tingling sensations, speech problems, aphasia, tinnitus, gastric stasis, pulsating or throbbing pain on one or both sides of the head, extreme sensitivity to light, sounds, or smells, worsening pain during physical activity, and vomiting, abdominal pain or heartburn, loss of appetite, lightheadedness, blurred vision, and fainting. Poor, insufficient, or non-response to triptan treatments results in the continued experience of one or more above symptoms. The treatment of such a patient with a CGRP antagonist, preferably ubrogepant or atogepant results in the improvement of reduced frequency or intensity of symptoms.

Preferably, the CGRP antagonist is selected from ubrogepant, atogepant, rimegepant or a pharmaceutically acceptable salt thereof.

In some embodiments, the CGRP antagonist is ubrogepant. In some embodiments, ubrogepant is administered at an oral dose of about 5 to about 500 mg once, twice or three times a day. In some embodiments, ubrogepant is administered at an oral dose of about 25 mg once, twice or three times a day. In some embodiments, ubrogepant is administered at an oral dose of about 50 mg once, twice or three times a day. In some embodiments, ubrogepant is administered at an oral dose of about 100 mg once, twice or three times a day. In some embodiments, ubrogepant is administered at an oral dose of about 200 mg once, twice or three times a day.

In one embodiment, ubrogepant is administered at a dose of about 1-1000 mg per day. In one embodiment, ubrogepant is administered at a dose of about 5, 10, 25, 50, 100, 200 or 400 mg per day.

In some embodiments the CGRP antagonist is atogepant. In some embodiments, atogepant is administered at an oral dose of about 5 to about 500 mg once, twice or three times a day. In some embodiments, atogepant is administered at an oral dose of about 25 mg once, twice or three times a day. In some embodiments, atogepant is administered at an oral dose of about 50 mg once, twice or three times a day. In some embodiments, atogepant is administered at an oral dose of about 100 mg once, twice or three times a day. In some embodiments, atogepant is administered at an oral dose of about 200 mg once, twice or three times a day.

In one embodiment, atogepant is administered at a dose of about 1-1000 mg per day. In one embodiment, atogepant is administered at a dose of about 5, 10, 15, 20, 25, 30, 40, 50, 60, 80, 100, 200, 250, 300, 400 or 500 mg per day.

In some embodiments, the CGRP antagonist is rimegepant. In some embodiments, rimegepant is administered at an oral dose of about 5 to about 500 mg once, twice or three times a day. In some embodiments, rimegepant is administered at an oral dose of about 25 mg once, twice or three times a day. In some embodiments, rimegepant is administered at an oral dose of about 50 mg once, twice or three times a day. In some embodiments, rimegepant is administered at an oral dose of about 100 mg once, twice or three times a day. In some embodiments, rimegepant is administered at an oral dose of about 200 mg once, twice or three times a day. In some embodiments, rimegepant is administered at an oral dose of about 5 to about 500 mg once, twice or three times a day. In some embodiments, rimegepant is administered at an oral dose of about 25 mg once, twice or three times a day. In some embodiments, rimegepant is administered at an oral dose of about 50 mg once, twice or three times a day. In some embodiments, rimegepant is administered at an oral dose of about 100 mg once, twice or three times a day. In some embodiments, rimegepant is administered at an oral dose of about 200 mg once, twice or three times a day.

In some embodiments, the CGRP-antagonist is an anti-calcitonin gene-related peptide receptor antibody (anti-CGRP antibody) or antigen-binding fragment thereof. For example, the antibody can be selected from galcanezumab, fremanezumab, eptinezumab or erenumab. In some embodiments, the anti-CGRP antibody or fragment thereof is administered at a dosage that is about 20% or 30% or 40% or 50% or 60% or 70% or 80% lower than the recommended dosage for the anti-CGRP antibody monotherapy for the treatment of migraine.

For example, erenumab can be administered weekly, biweekly, monthly, every two months, every three months, every four months, every five months or every six months at a dosage of about 5 mg to about 500 mg. Erenumab can be administered parenterally, subcutaneously or by peripheral administration. (Brauser D., Phase 3 STRIVE and ARISE Trials Show Efficacy, Safety for Erenumab in Migraine Prevention, Medscape Medical News, 2017). In one embodiment, erenumab can be administered subcutaneously at a dose of about 5 mg to about 500 mg every one, two, three, four, five, six, seven, eight, nine or ten weeks. In one embodiment, erenumab can be administered subcutaneously at a dose of about 10 mg to about 200 mg every one, two, three, four, five, six, seven, eight, nine or ten weeks. In one embodiment, erenumab can be administered subcutaneously at a dose of about 25 mg to about 150 mg every one, two, three, four, five, six, seven, eight, nine or ten weeks. In one embodiment, erenumab can be administered subcutaneously at a dose of about 90 mg to about 120 mg every one, two, three, four, five, six, seven, eight, nine or ten weeks. In one embodiment, erenumab can be administered subcutaneously at a dose of about 50 mg to about 60 mg every one, two, three, four, five, six, seven, eight, nine or ten weeks. In one embodiment, erenumab can be administered subcutaneously at a dose of about 70 mg every one, two, three, four, five, six, seven, eight, nine or ten weeks. In one embodiment, erenumab can be administered subcutaneously at a dose of about 140 mg every one, two, three, four, five, six, seven, eight, nine or ten weeks. In one embodiment, erenumab can be administered subcutaneously at a monthly dose of about 140 mg. In one embodiment, erenumab can be administered subcutaneously at a monthly dose of about 70 mg. In one embodiment, erenumab can be administered subcutaneously at a dose of about 140 mg every two months. In one embodiment, erenumab can be administered subcutaneously at a dose of about 70 mg every two months. In one embodiment, erenumab can be administered subcutaneously at a dose of about 140 mg every three months. In one embodiment, erenumab can be administered subcutaneously at a dose of about 70 mg every three months.

In one embodiment, an anti-CGRP antibody galcanezumab can be administered weekly, biweekly, monthly, every two months, every three months, every four months, every five months or every six months at a dosage of about 5 mg to about 500 mg. In one embodiment, galcanezumab is administered subcutaneously at a dose of about 10 mg to about 500 mg every one, two, three, four, five, six, seven, eight, nine or ten weeks. In one embodiment, galcanezumab is administered subcutaneously at a dose of about 50 mg to about 300 mg every one, two, three, four, five, six, seven, eight, nine or ten weeks. In one embodiment, galcanezumab is administered subcutaneously at a dose of about 75 mg to about 250 mg every one, two, three, four, five, six, seven, eight, nine or ten weeks. In one embodiment, galcanezumab is administered subcutaneously at a dose of about 75 mg to about 100 mg every one, two, three, four, five, six, seven, eight, nine or ten weeks. In one embodiment, galcanezumab is administered subcutaneously at a dose of about 150 mg to about 220 mg every one, two, three, four, five, six, seven, eight, nine or ten weeks. In one embodiment, galcanezumab is administered subcutaneously at a dose of about 120 mg every one, two, three, four, five, six, seven, eight, nine or ten weeks. In one embodiment, galcanezumab is administered subcutaneously at a dose of about 240 mg every one, two, three, four, five, six, seven, eight, nine or ten weeks. In one embodiment, galcanezumab is administered subcutaneously at a monthly dose of about 240 mg. In one embodiment, galcanezumab is administered subcutaneously at a monthly dose of about 120 mg. In one embodiment, galcanezumab is administered subcutaneously at a dose of about 240 mg every two months. In one embodiment, galcanezumab is administered subcutaneously at a dose of about 120 mg every two months. In one embodiment, galcanezumab is administered subcutaneously at a dose of about 240 mg every three months. In one embodiment, galcanezumab is administered subcutaneously at a dose of about 120 mg every three months.

In one embodiment, fremanezumab is administered subcutaneously at a dose of about 100 mg to about 1000 mg every one, two, three, four, five, six, seven, eight, nine or ten weeks. In one embodiment, fremanezumab is administered subcutaneously at a dose of about 150 mg to about 700 mg every one, two, three, four, five, six, seven, eight, nine or ten weeks. In one embodiment, fremanezumab is administered subcutaneously at a dose of about 150 mg to about 500 mg every one, two, three, four, five, six, seven, eight, nine or ten weeks. In one embodiment, fremanezumab is administered subcutaneously at a dose of about 150 mg to about 200 mg every one, two, three, four, five, six, seven, eight, nine or ten weeks. In one embodiment, fremanezumab is administered subcutaneously at a dose of about 150 mg to about 500 mg every one, two, three, four, five, six, seven, eight, nine or ten weeks.

In one embodiment, fremanezumab is administered subcutaneously at a dose of about 225 mg every one, two, three, four, five, six, seven, eight, nine or ten weeks. In one embodiment, fremanezumab is administered subcutaneously at a dose of about 450 mg every one, two, three, four, five, six, seven, eight, nine or ten weeks. In one embodiment, fremanezumab is administered subcutaneously at a dose of about 675 mg every one, two, three, four, five, six, seven, eight, nine or ten weeks. In one embodiment, fremanezumab is administered subcutaneously at a monthly dose of about 225 mg. In one embodiment, fremanezumab is administered subcutaneously at a monthly dose of about 450 mg. In one embodiment, fremanezumab is administered subcutaneously at a monthly dose of about 675 mg. In one embodiment, fremanezumab is administered subcutaneously at a dose of about 225 mg every two months. In one embodiment, fremanezumab is administered subcutaneously at a dose of about 450 mg every two months. In one embodiment, fremanezumab is administered subcutaneously at a dose of about 225 mg every three months. In one embodiment, fremanezumab is administered subcutaneously at a dose of about 450 mg every three months. In one embodiment, fremanezumab is administered subcutaneously at a dose of about 675 mg every three months.

In one embodiment, eptinezumab is administered subcutaneously at a dose of about 50 mg to about 1000 mg every one, two, three, four, five, six, seven, eight, nine or ten weeks. In one embodiment, eptinezumab is administered subcutaneously at a dose of about 100 mg to about 700 mg every one, two, three, four, five, six, seven, eight, nine or ten weeks. In one embodiment, eptinezumab is administered subcutaneously at a dose of about 200 mg to about 500 mg every one, two, three, four, five, six, seven, eight, nine or ten weeks. In one embodiment, eptinezumab is administered subcutaneously at a dose of about 250 mg to about 350 mg every one, two, three, four, five, six, seven, eight, nine or ten weeks. In one embodiment, eptinezumab is administered subcutaneously at a dose of about 300 mg every one, two, three, four, five, six, seven, eight, nine or ten weeks. In one embodiment, eptinezumab is administered subcutaneously at a monthly dose of about 100 mg. In one embodiment, eptinezumab is administered subcutaneously at a monthly dose of about 200 mg. In one embodiment, eptinezumab is administered subcutaneously at a monthly dose of about 300 mg. In one embodiment, eptinezumab is administered subcutaneously at a dose of about 100 mg every two months. In one embodiment, eptinezumab is administered subcutaneously at a dose of about 200 mg every two months. In one embodiment, eptinezumab is administered subcutaneously at a dose of about 300 mg every two months. In one embodiment, eptinezumab is administered subcutaneously at a dose of about 100 mg every three months. In one embodiment, eptinezumab is administered subcutaneously at a dose of about 200 mg every three months. In one embodiment, eptinezumab is administered subcutaneously at a dose of about 300 mg every three months.

In some embodiments, the CGRP-antagonist can be administered orally, sublingually, transdermally, subcutaneously, intravenously, or intramuscularly.

### Definitions

As used herein, the words or terms set forth below have the following definitions:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“CGRP”, abbreviated for Calcitonin-Gene-Related-Peptide, as used herein encompasses any member of the calcitonin family, including any calcitonin gene related peptide and analogs, calcitonin, amylin, adrenomedullin and their analogs.

“CGRP antagonist” refers to any molecule that exhibits any one or more of the following characteristics: (a) bind to CGRP or CGRP-R and the binding results in a reduction or inhibition of CGRP activity; (b) block CGRP from binding to its receptor(s); (c) block or decrease CGRP receptor activation; (d) inhibit CGRP biological activity or downstream pathways mediated by CGRP signaling function; (e) increase clearance of CGRP; and (f) inhibit or reduce CGRP synthesis, production or release. CGRP antagonists include but are not limited to antibodies to CGRP, antibodies to the CGRP-R, small molecules that antagonize CGRP, and small molecules that antagonize CGRP-R.

“Effective amount” as applied to the biologically active ingredient means that amount of the ingredient which is generally sufficient to effect a desired change in the subject. For example, where the desired effect is a reduction in an autoimmune disorder symptom, an effective amount of the ingredient is that amount which causes at least a substantial reduction of the autoimmune disorder symptom, and without resulting in significant toxicity.

“Intramuscular” or “intramuscularly” means into or within (as in administration or injection of a CGRP antagonist into) a muscle.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's skin.

“Patient” means a human or non-human subject receiving medical or veterinary care. Accordingly, the compositions as disclosed herein can be used in treating any animal, such as, for example, mammals, or the like.

“Peripherally administering” or “peripheral administration” means subdermal, intradermal, transdermal, or subcutaneous administration, but excludes intramuscular administration. “Peripheral” means in a subdermal location, and excludes visceral sites.

“Pharmaceutical composition” means a composition comprising an active pharmaceutical ingredient, such as, for example, a CGRP antagonist, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

The constituent ingredients of a pharmaceutical composition can be included in a single composition (that is, all the constituent ingredients, except for any required reconstitution fluid, are present at the time of initial compounding of the pharmaceutical composition) or as a two-component system, for example a vacuum-dried composition reconstituted with a reconstitution vehicle which can, for example, contain an ingredient not present in the initial compounding of the pharmaceutical composition. A two-component system can provide several benefits, including that of allowing incorporation of ingredients which are not sufficiently compatible for long-term shelf storage with the first component of the two-component system. A pharmaceutical composition can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

“Tonicity agent” means a low molecular weight excipient which is included in a formulation to provide isotonicity. Disaccharide, such as trehalose or sucrose, polyalcohol, such as sorbitol or mannitol, monosaccharide, such as glucose, and salt, such as sodium chloride, can serve as a tonicity agent.

“Polysaccharide” means a polymer of more than two saccharide molecule monomers. The monomers can be identical or different.

“Stabilizers” can include excipients, and can include protein and non-protein molecules.

“Therapeutic formulation” means a formulation can be used to treat and thereby alleviate a disorder or a disease, such as, for example, a disorder or a disease characterized by hyperactivity (i.e. spasticity) of a peripheral muscle.

“Treating” means to alleviate (or to eliminate) at least one symptom of a condition or disorder, such as, for example, wrinkles, spasticity, depression, pain (such as, for example, headache pain), bladder overactivity, or the like, either temporarily or permanently.

### Examples

The following non-limiting examples provide those of ordinary skill in the art with possible case scenarios and specific methods to treat conditions within the scope of the present disclosure and are not intended to limit the scope of the disclosure.

Study A and Study B were multicenter, randomized, double-blind, placebo-controlled, parallel-group, studies designed to evaluate the efficacy, safety, and tolerability of three doses of ubrogepant (25 mg, 50 mg and 100 mg) compared to placebo for the acute treatment of a single migraine attack. In Study A, patients were randomized (1:1:1) to 1 of 3 treatment groups: placebo, ubrogepant 50 mg, or ubrogepant 100 mg. In Study B, patients were randomized (1:1:1) to 1 of 3 treatment groups: placebo, ubrogepant 25 mg, or ubrogepant 50 mg. Patients were stratified by their previous response to triptans (triptan responder, triptan insufficient responder, triptan naïve) and their current use of prophylactic concomitant medication for migraine (yes/no).

To be randomized, eligible patients had to be 18 to 75 years of age (inclusive), have a history of migraine with or without aura for at least 1 year consistent with a diagnosis according to the International Classification of Headache Disorders criteria, 3rd edition, beta version, and had to have experienced between 2 to 8 migraine attacks with moderate to severe headache pain in each of the 3 months before Screening (Visit 1). Patients who had clinically significant hematologic, endocrine, cardiovascular, cerebrovascular, pulmonary, renal, hepatic, gastrointestinal, or neurologic disease were excluded from the study. Study patients randomized to a treatment group had up to 60 days to treat a single qualifying migraine attack of moderate or severe pain intensity at home. In Study A, a total of 1672 patients were randomized to double-blind treatment (ITT population), and 1436 patients took at least 1 dose of double-blind IP (safety population). A total of 1327 treated patients recorded a baseline migraine headache severity measurement and at least 1 postdose migraine headache severity or migraine-associated symptom measurement within 2 hours after dosing (mITT population). In Study B, a total of 1,686 patients were randomized to double-blind treatment (ITT population), and 1,465 patients took at least 1 dose of double-blind IP (safety population). A total of 1355 treated patients recorded a baseline migraine headache severity measurement and at least 1 postdose migraine headache severity or migraine-associated symptom measurement within 2 hours after dosing (mITT population).

The coprimary efficacy endpoints for the United States were pain freedom (PF) at 2 hours after the initial dose, defined as a reduction in headache severity from moderate/severe at baseline to no pain, at 2 hours after the initial dose and absence of the most bothersome migraine-associated symptom at 2 hours after the initial dose. The most bothersome migraine-associated symptom was identified at baseline for each patient.

Results from Study A and Study B are presented in tables below:

TABLE 1
Study B: Number of Patients in Each Triptan Response Category (mITT population)

| | | | Ubrogepant | Ubrogepant |
|---|---|---|---|---|
| Triptan Response | Placebo | Ubrogepant 25 mg | 50 mg | Total |
| Category, n (%) | (N = 456) | (N = 435) | (N = 464) | (N = 1355) |
| Triptan Responder | 159 (34.9) | 151 (34.7) | 160 (34.5) | 470 (34.7) |
| Triptan Insufficient | 106 (23.2) | 100 (23.0) | 110 (23.7) | 316 (23.3) |
| Responder* | | | | |
| Insufficient Efficacy | 81 (76.4) | 87 (87.0) | 92 (83.6) | 260 (82.3) |
| Insufficient | 20 (18.9) | 12 (12.0) | 15 (13.6) | 47 (14.9) |
| Tolerability | | | | |
| Contraindications, | 3 (2.8) | 1 (1.0) | 2 (1.8) | 6 (1.9) |
| Warnings/Precautions | | | | |
| Triptan Naive | 191 (41.9) | 184 (42.3) | 194 (41.8) | 569 (42.0) |

*A Triptan Insufficient Responder was is a study participant who meets any of the following criteria:
(i) currently uses a triptan or has used a triptan in the past 6 months, and on the occasions that a triptan dose was taken, has not achieved pain freedom (no headache pain) at 2 hours postdose on more than half of those occasions;
(ii) no longer uses a triptan due to lack of efficacy;
(iii) no longer uses a triptan due to side effects; or
(iv) never used a triptan due to warnings, precautions, or contraindications.

TABLE 2
Study B Sub-group Analysis: Co-Primary Endpoint-Pain Freedom at
2 hours after the initial dose by historical triptan response (mITT Population)

| | Placebo | Ubrogepant 25 mg | Ubrogepant 50 mg |
|---|---|---|---|
| Subgroup Statistics | (N = 456) | (N = 435) | (N = 464) |
| Triptan Insufficient | | | |
| Responder | | | |
| N1 | 106 | 100 | 110 |
| Responder, n | 11 (10.4) | 18 (18.0) | 21 (19.1) |
| Odds Ratio (95% CI) | | 1.81 (0.80, 4.08) | 1.97 (0.89, 4.34) |
| Triptan Responder | | | |
| N1 | 159 | 151 | 160 |
| Responder, n | 18 (11.3) | 25 (16.6) | 35 (21.9) |
| Odds Ratio (95% CI) | | 1.52 (0.79, 2.93) | 2.19 (1.18, 4.09) |
| Triptan Naïve | | | |
| N1 | 191 | 184 | 194 |
| Responder, n | 36 (18.8) | 47 (25.5) | 45 (23.2) |
| Odds Ratio (95% CI) | | 1.50 (0.91, 2.48) | 1.22 (0.74, 2.01) |
| Treatment-by-subgroup | 0.511 | | |
| interaction p-value | | | |

TABLE 3
Study B Sub-group Analysis: Co-Primary Endpoint-Absence of
Most-Bothersome Migraine Associated Symptom at 2 hours after
the initial dose by historical triptan response (mITT Population)

| | Placebo | Ubrogepant 25 mg | Ubrogepant 50 mg |
|---|---|---|---|
| Subgroup Statistics | (N = 456) | (N = 435) | (N = 464) |
| Triptan Insufficient | | | |
| Responder | | | |
| N1 | 106 | 100 | 110 |
| Responder, n | 26 (24.5) | 37 (37.0) | 42 (38.2) |
| Odds Ratio (95% CI) | | 1.65 (0.90, 3.05) | 1.72 (0.95, 3.13) |
| Triptan Responder | | | |
| N1 | 159 | 150 | 160 |
| Responder, n | 43 (27.0) | 48 (32.0) | 62 (38.8) |
| Odds Ratio (95% CI) | | 1.26 (0.77, 2.07) | 1.76 (1.09, 2.85) |
| Triptan Naïve | | | |
| N1 | 191 | 184 | 193 |
| Responder, n | 56 (29.3) | 63 (34.2) | 76 (39.4) |
| Odds Ratio (95% CI) | | 1.32 (0.84, 2.05) | 1.54 (1.00, 2.37) |
| Treatment-by-subgroup | 0.9251 | | |
| interaction p-value | | | |

TABLE 4
Study A: Number of Patients in Each Triptan Response Category in the mITT population

| | | Ubrogepant | Ubrogepant | Ubrogepant |
|---|---|---|---|---|
| Triptan Response | Placebo | 50 mg | 100 mg | Total |
| Category n (%) | (N = 456) | (N =423) | (N = 448) | (N = 1327) |
| Triptan Responder | 191 (41.9) | 172 (40.7) | 173 (38.6) | 536 (40.4) |
| Triptan Insufficient | 117 (25.7) | 118 (27.9) | 126 (28.1) | 361 (27.2) |
| Responder | | | | |
| Insufficient Efficacy | 93 (79.5) | 93 (78.8) | 97 (77.0) | 283 (78.4) |
| Insufficient Tolerability | 18 (15.4) | 22 (18.6) | 24 (19.0) | 64 (17.7) |
| Contraindications, | 4 (3.4) | 3 (2.5) | 4 (3.2) | 11 (3.0) |
| Warnings/Precautions | | | | |
| Triptan Naive | 148 (32.5) | 133 (31.4) | 149 (33.3) | 430 (32.4) |

TABLE 5
Study A Sub-group Analysis: Co-Primary Endpoint-Pain Freedom at
2 hours after the initial dose by historical triptan response (mITT Population)

| | Placebo | Ubrogepant 50 mg | Ubrogepant 100 mg |
|---|---|---|---|
| Subgroup Statistics | (N = 456) | (N = 423) | (N = 448) |
| Triptan Insufficient | | | |
| Responder | | | |
| N1 | 117 | 118 | 126 |
| Responder, n | 7 (6.0) | 16 (13.6) | 18 (14.3) |
| Odds Ratio (95% CI) | | 2.41 (0.95, 6.12) | 2.49 (1.00, 6.22) |
| Triptan Responder | | | |
| N1 | 191 | 171 | 173 |
| Responder, n | 22 (11.5) | 32 (18.7) | 46 (26.6) |
| Odds Ratio (95% CI) | | 1.86 (1.03, 3.36) | 2.89 (1.65, 5.06) |
| Triptan Naïve | | | |
| Ni | 148 | 133 | 149 |
| Responder, n | 25 (16.9) | 33 (24.8) | 31 (20.8) |
| Odds Ratio (95% CI) | | 1.60 (0.89, 2.88) | 1.26 (0.70, 2.28) |
| Treatment-by-subgroup | 0.2561 | | |
| interaction p-value | | | |

TABLE 6
Study A Sub-group Analysis: Co-Primary Endpoint-Absence of
Most-Bothersome Migraine Associated Symptom at 2 hours after
the initial dose by historical triptan response (mITT Population)

| | Placebo | Ubrogepant 50 mg | Ubrogepant 100 mg |
|---|---|---|---|
| Subgroup Statistics | (N = 456) | (N = 423) | (N = 448) |
| Triptan Insufficient | | | |
| Responder | | | |
| N1 | 117 | 116 | 126 |
| Responder, n | 26 (22.2) | 39 (33.6) | 38 (30.2) |
| Odds Ratio (95% CI) | | 1.80 (1.00, 3.25) | 1.50 (0.84, 2.70) |
| Triptan Responder | | | |
| N1 | 190 | 172 | 173 |
| Responder, n | 51 (26.8) | 67 (39.0) | 75 (43.4) |
| Odds Ratio (95% CI) | | 1.85 (1.18, 2.90) | 2.18 (1.40, 3.41) |
| Triptan Naïve | | | |
| N1 | 147 | 132 | 149 |
| Responder, n | 49 (33.3) | 56 (42.4) | 56 (37.6) |
| Odds Ratio (95% CI) | | 1.48 (0.90, 2.42) | 1.22 (0.75, 1.98) |
| Treatment-by- | 0.4938 | | |
| subgroup interaction | | | |
| p-value | | | |

1. A method of treating migraine comprising the step of administering an effective amount of a CGRP antagonist, or a pharmaceutically acceptable salt thereof to a patient in need thereof, wherein said patient is a non-responder, infrequent responder or an insufficient responder to one or more triptan drugs.

2. The method according to claim 1, wherein said CGRP antagonist is ubrogepant, atogepant, or a pharmaceutically acceptable salt thereof.

3. The method according to claim 1 or 2 wherein said triptan drug is selected from rizatriptan, sumatriptan, naratriptan, eletriptan, donitriptan, almotriptan, frovatriptan, avitriptan and zolmitriptan.

4. The method according to any of claims 1-3 wherein said patient had prior treatment with one or more triptans for a period of one, two, three, four, five, six, seven, eight, nine, ten, eleven, twelve weeks or more.

5. The method according to any of the above claims wherein said patient experiences reduced frequency or reduced severity of migraine after treatment with ubrogepant or atogepant.

6. The method according to any of the above claims wherein said patient suffers from one or more symptoms of migraine selected from sinusitis, nausea, nasopharangytis, photophobia, appetite changes, cognition and concentration difficulties, cold extremities, diarrhea or other bowel changes, excitement or irritability, fatigue, frequent urination, memory changes, weakness, yawning, stretching, seeing bright spots or flashes of light, vision loss, seeing dark spots, tingling sensations, speech problems, aphasia, tinnitus, gastric stasis, pulsating or throbbing pain on one or both sides of the head, extreme sensitivity to light, sounds, or smells, worsening pain during physical activity, and vomiting, abdominal pain or heartburn, loss of appetite, lightheadedness, blurred vision, and fainting.

7. The method according to claim 6 wherein said one or more symptoms of migraine is present after the treatment with one or more triptan drugs.

8. The method according to claim 7 wherein said one or more symptoms of migraine is reduced after treatment with ubrogepant or atogepant.

9. The method according to any of the above claims wherein ubrogepant is administered at a dose of about 1-1000 mg per day.

10. The method according to any of the above claims wherein ubrogepant is administered at a dose of about 5, 10, 25, 50, 100, 200 or 400 mg per day.

11. The method according to any of claims 2-8 wherein atogepant is administered at a dose of about 1-1000 mg per day.

12. The method according to any of claims 2-8 wherein atogepant is administered at a dose of about 5, 10, 15, 20, 25, 30, 40, 50, 60, 80, 100, 200, 250, 300, 400 or 500 mg per day.
